(19) United States
(12) Patent Application Publication
(10) Pub. No.: US 2007/0260329 A1
(43) Pub. Date: Nov. 8, 2007

(54) METHOD TO DETERMINE FITTING HEIGHT OF PROSTHESIS

(75) Inventor: Christian Hiemisch, Duderstadt (DE)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: Otto Bock Healthcare IP GmbH & Co., KG, Duderstadt (DE)

(21) Appl. No.: 11/744,094

(22) Filed: May 3, 2007

(30) Foreign Application Priority Data
May 3, 2006 (DE) 10 2006 021 064.6

(51) Int. Cl. A61F 2/62; A61F 2/76

(52) U.S. Cl. 623/38; 623/912

(57) ABSTRACT

Embodiments of the present invention relate to determining the overall height of a prosthesis composed of several components, in which at least some of the components can be adjustably connected to one another via adapters using a substantially spherical surfaces. In certain embodiments, each component can be assigned a system height value and for some components the system height values are adjustable by means of a substantially spherical surface. In other embodiments, a midpoint dimension can be assigned in the respective substantially spherical surface, and these system height values for the components mounted on one another can be added together to determine the overall height.

This patent application claims priority from German patent Application No. 10 2006 021 064.6, entitled “VERFAHREN ZUR ERMITTLUNG DER EINBAUHÖHE EINER PROTHESE, filed on May 3, 2006 incorporated herein by reference in its entirety.

### TECHNICAL FIELD

Embodiments of the present invention relate to methods for determining the height of a prosthesis device. In certain embodiments, the prosthesis is composed of several components, one or more of which can be adjustably connected to one or more other components via adapters. In certain particular embodiments, a prosthesis contemplated herein can include adapters and substantially spherical surfaces for adjusting height of prosthesis.

### BACKGROUND

Prostheses are customarily designed as modular systems in order to allow assembly that conforms to the requirements of a patient. For example, a below-the-knee prosthesis may need a shaft for adjustably fitting a below-knee tube where the tube is connected to a foot module using an adapter. In this example, the foot module can be designed to meet requirements of a specific patient to increase walking comfort, sports activities, etc.

In addition, a prosthesis can be used for a patient whose leg has been amputated above the knee. Here, a knee joint is also used where the joint can be arranged to the height of the natural knee of the intact leg of a particular patient.

The overall height of a prosthesis assembled as described above is determined by measuring the prosthesis and adapting an optionally length-adjustable individual part, for example, a length-adjustable tube part of the below-knee prosthesis. Measuring the prosthesis and determining the overall height required for the patient necessitates the involvement of a person experienced in orthopedic mechanics and with many of these situations the adjustment is often done inaccurately, even though the required overall height can be calculated with sufficient precision from patient data. Thus, a need exists for a more readily adjustable prosthesis that does not require a person experienced in orthopedic mechanics.

### SUMMARY

Embodiments of the present invention provide for methods for determining and adjusting overall height of prosthesis composed of several components, that is easier and less susceptible to error.

Certain embodiments concern assigning each component of prosthesis a system height value which, for components that are adjustable by a substantially spherical surface, can include a dimension reaching as far as the midpoint of the respective substantially spherical surface. In accordance with these embodiments, these system height values for the components can be mounted on one another and then added together to determine overall height.

Embodiments herein are based on the fact that determining the effective overall heights of the components of prosthesis is often problematic and has given rise to the incorrect measurements. Accordingly, embodiments herein concern each component used to construct a prosthesis be assigned a system height value which, for those components that are designed to be adjustable via a substantially spherical surface, includes a virtual dimension, for example, a dimension that is capable of reaching as far as the midpoint of the respective substantially spherical surface. By using system height values, predetermined connectable component dimensions can be added, because the components designed to be adjustably connected to one another via a substantially spherical surface have a coincident midpoint of the respective sphere surfaces. The system height value can thus be assigned to the component during its production, such that only addition of the system height values is needed in order to determine the effective overall height of the prosthesis.

In certain embodiments contemplated herein, some components can have a negative system height value, for example, a concavely shaped shaft holder in which the sphere midpoint lies within the shaft or the amputation stump. In this example, the negative value of the system height has to be included in order to arrive at the effective overall height of the prosthesis.

Other embodiments concern methods for determining system height of prosthesis to the point of a previously selected reference line, for example, determining the knee gap height of the intact leg of a patient, in order to ensure that the prosthesis extending to the thigh has a knee joint that is arranged at a corresponding height relative to the intact leg of the patient. This can be performed in order to assure the patient has as natural a gait as possible.

In certain embodiments, a modular prosthesis generally includes at least one length-adjustable component, which can be provided with a length setting that can be read off of the component. In accordance with these embodiments, it is expedient if the length setting that can be read off is incorporated in direct proportion to the system height value, in order to determine an accurate system height value of the length-adjustable component

### BRIEF DESCRIPTION OF THE DRAWINGS

The following drawings form part of the present specification and are included to further demonstrate certain embodiments disclosed herein. Embodiments may be better understood by reference to one or more of these drawings in combination with the detailed description of specific embodiments presented herein.

FIG. 1a represents an exemplary below-knee prosthesis component, a foot module.

FIG. 1b represents an exemplary below-knee prosthesis component, a below-knee module.

FIG. 1c represents an exemplary below-knee prosthesis component, a shaft holder.

FIG. 2a represents the exemplary component of the below-knee prosthesis represented in FIG. 1a, with system height values.

FIG. 2b represents the exemplary components of the below-knee prosthesis represented in FIG. 1b, with system height values.

FIG. 2c represents the exemplary components of the below-knee prosthesis represented in FIG. 1c, with system height values.

FIG. 3 represents an exemplary assembled prosthesis.

FIG. 4 represents a schematic of an example of one above-knee prosthesis.

### DETAILED DESCRIPTION

In the following sections, various exemplary methods are described in order to detail various embodiments of the invention. It will be obvious to one skilled in the art that practicing the various embodiments does not require the employment of all or even some of the specific details outlined herein, but rather other specific details may be modified. In some cases, well known methods, or components have not been included in the description.

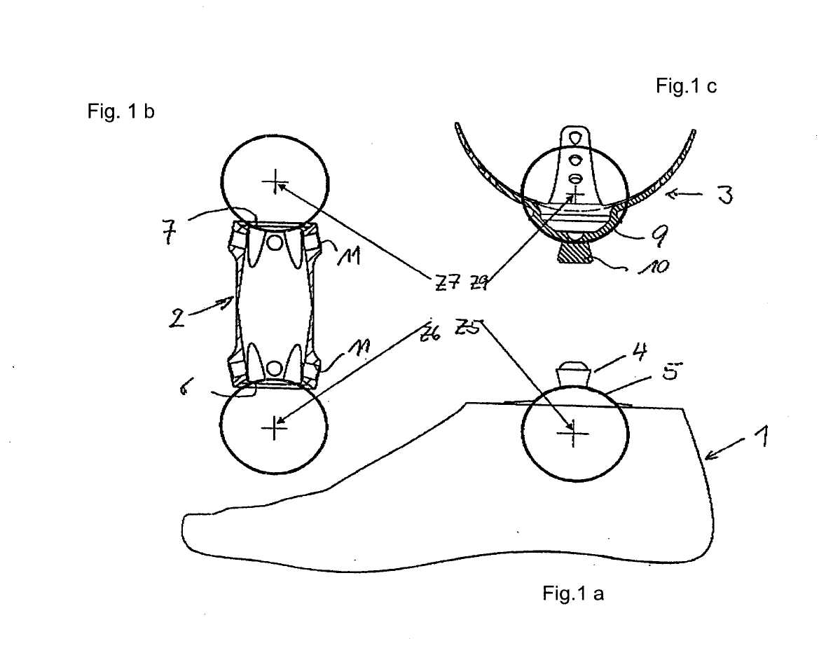

Certain embodiments of the present invention concern simplifying adjustments of a prosthesis, to better suit a patient, using methods disclosed herein. An example prosthesis is represented in FIGS. 1a, 1b & 1c. The prosthesis represented in FIGS. 1a, 1b, 1c, is composed of three components, a foot module 1 in FIG. 1a, a below-knee module 2 in FIG. 1b, and a shaft holder 3 in FIG. 1c.

FIG. 1a illustrates an exemplary foot module 1 having an adapter 4 that has a substantially spherical adjustment surface 5.

In this exemplary module, a below-knee module 2 can be connected to the foot module 1 by means of the adapter 4, said below-knee module 2 having, at its lower end, a concave spherical adjustment surface 6 capable of interacting with the substantially spherical adjustment surface 5.

In addition, the exemplary module of FIG. 1b, the below-knee module 2 has a corresponding concave spherical adjustment surface 7 at the upper end that has substantially the same sphere radius as a spherical (convex) adjustment surface 9 of an adapter 10 of a shaft holder 3.

In one example, the adapters 4, 10 have substantially the shape of an inverted frustum of a pyramid with adjustment surfaces capable of being adjusted with adjustment screws (not shown), the latter interacting in threaded bores 11 of the adapter counter-pieces at the ends of the below-knee prosthesis 2.

In further embodiments, the substantially spherical adjustment surfaces 5, 6, 7, 9 can each have associated sphere midpoints Z5, Z6, Z7, Z9.

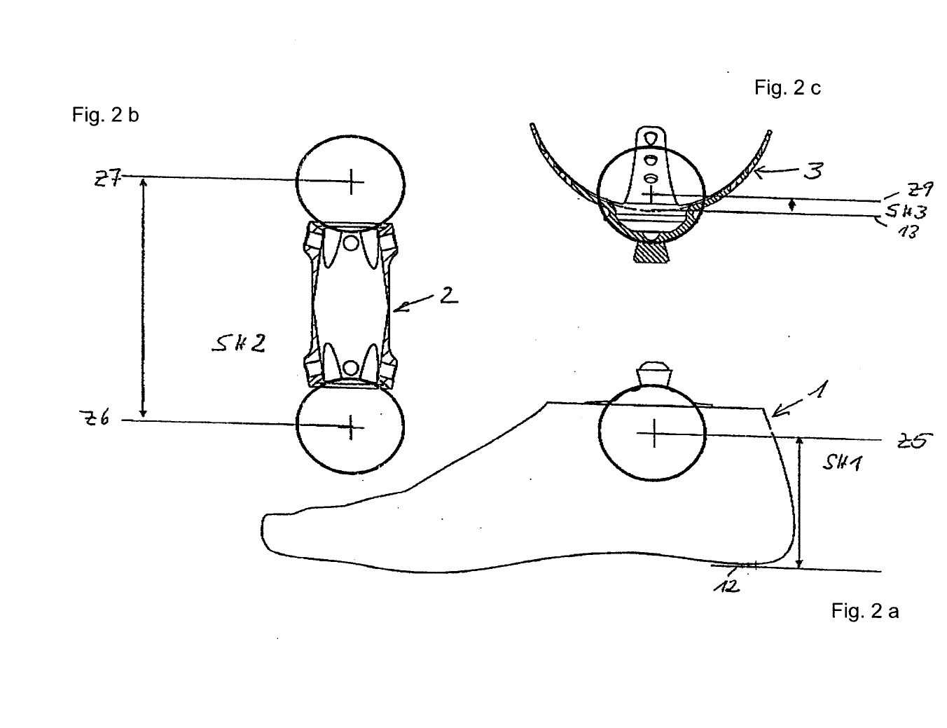

Certain embodiments herein concern correctly assembling prosthesis components 1, 2, 3, where sphere midpoints Z5 and Z6 correspond to one another and the sphere midpoints Z7 and Z9 correspond to one another. In accordance with these embodiments, components 1, 2, 3 can be assigned system height values SH1, SH2 and SH3 in accordance with FIGS. 2a, 2b, 2c.

In FIG. 2a, the system height value SH1 of the foot module 1 can be derived from the distance between a tread surface 12 of the foot module 1 and the height of the sphere midpoint Z5.

In one illustrative embodiment, the system height SH1 is 72 mm.

In another embodiment, the system height value SH2 for the below-knee module 2 can be derived from the distance between the sphere midpoints Z6 and Z7. In one illustrative embodiment, the system height SH2 is 112 mm.

Other examples include that the system height SH3 can be derived from the distance between the sphere midpoint Z9 and a shaft end height 13 of a shaft (not shown) which is received by the shaft holder 3 to extend from an amputation stump.

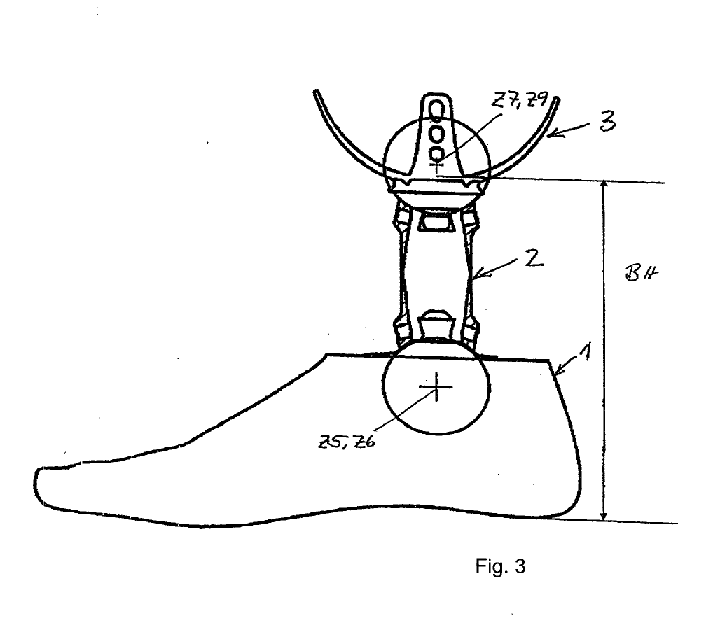

In one particular embodiment, the sphere midpoint Z9 of FIGS. 2c and 3 lies above the shaft end height 13, here the value for the system height SH3 is negative. In one illustrative embodiment, the system height SH3 is −5 mm.

FIG. 3 illustrates an exemplary prosthesis composed of components 1, 2 & 3. The sphere midpoints Z5 and Z6 and the corresponding sphere midpoints Z7 and Z9 are capable of coinciding when the prosthesis is assembled. A relevant overall height BH of the prosthesis is derived from the addition of the system heights SH1+SH2+SH3. In this particular illustrative embodiment, the overall height BH can be derived by 72+112−5 mm=179 mm.

Thus, the relevant overall height BH can be determined in a simple manner by the system heights SH1, SH2 and SH3 assigned to the components 1, 2, 3.

In accordance with these embodiments, where one component can be designed to be adjustable in length, having for example, a telescopically displaceable tube section, the adjustment can be read off the tube section. Here, the component can be assigned a standard system height which can be modified by the readable length setting through addition or subtraction of a displayed deviation value from a starting position, resulting in the relevant value for the system height SH after the length adjustment.

This exemplary embodiment represented here was based on an exemplary below-knee prosthesis for a patient whose leg has been amputated below the knee joint. If a prosthesis is constructed for a patient whose leg has been amputated above the knee joint, for example, through the femur, the problem of determining the overall height BH for the shaft end height arises, and this prosthesis further requires that the knee joint is arranged at a suitable height.

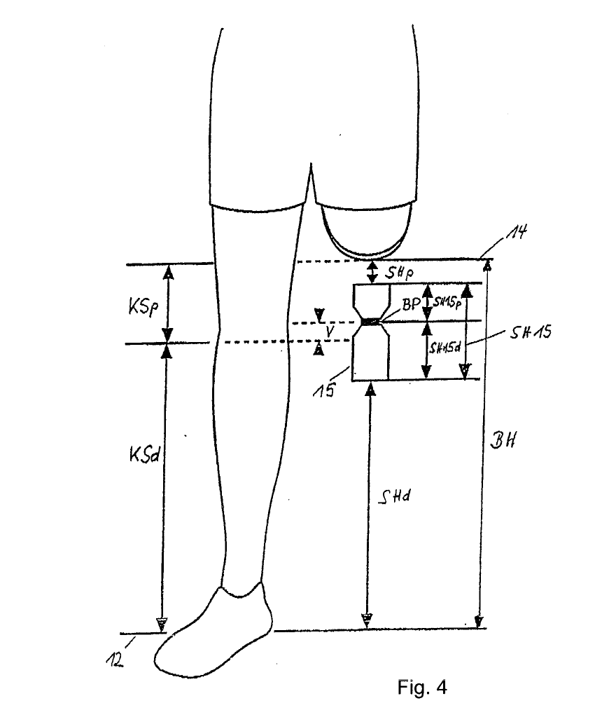

Another exemplary embodiment is represented in FIG. 4. FIG. 4 represents the knee gap height KSd between the knee gap of the intact leg and the tread surface 12 determined as reference height. The overall height BH can be determined by the sum of the knee gap height KSd and a distance KSp between the knee gap and a shaft end height 14 of a shaft (not shown here) receiving the thigh amputation stump.

In addition, FIG. 4 represents a schematic of a knee joint 15, where reference point BP can be determined. For the knee joint 15, a system height SH15 can also be determined similar to components 1, 2, 3. The system height SH15 of the knee joint 15 can be subdivided into a proximal system height SH15p and a distal system height SH15d, which are capable of interacting at the height of the reference point BP.

In another embodiment, a monocentric knee joint, which is constructed with a positionally fixed pivot axis, reference point BP can be derived from the height of the pivot axis. In another embodiment, a polycentric knee joint, for example, in a four-bar arrangement, reference point BP can be determined by the upper front hinge. It can be beneficial to arrange the reference point BP with an offset V above the knee gap height KSd, for example, approximately 20 mm above the knee gap height KSd.

In one particular embodiment, for knee joint 15, a total system height SH15 can be determined, and, based on its reference point BP in relation to the knee gap height KSd of the intact leg, it is possible to calculate a proximal system height SHp and a distal system height SHd. These system heights SHp and SHd can then be finalized with the system heights of the component presented for the construction of the above-knee prosthesis, for example, by using at least one length-adjustable component. When determining the system height SH15 of the knee joint, whether the knee joint 15 is terminated on its top face and bottom face with a spherical adjustment surface might be considered, which can result in an optionally virtual dimension SH15 for the system height, as detailed based on the prior figures.

What is claimed is:

1. A method for determining overall height of prosthesis comprising:
obtaining a prosthesis comprising several components;
assigning a system height value to each component;
adjustably connecting two or more of the components using adapters;
using substantially spherical surfaces for adjusting system height values for some of the adjustable components;
determining a midpoint of the spherical surfaces;
assembling the components to form the prosthesis; and
adding the system height values of predetermined components to identify overall height of the prosthesis.

2. The method of claim 1, wherein an individual component having an adjustable length is assigned a system height value directly proportional to the respective length setting.

3. The method of claim 1, wherein at least one component having a system height value is further divided into subsidiary system height values with respect to a reference point in the prosthesis component.

4. The method of claim 3, further comprising orienting the reference point of the component of the prosthesis in relation to height of a reference point in an intact limb.

5. The method of claim 4, wherein the reference point of the intact limb is the knee gap of the knee joint.

5. The method of claim 5, further comprising the reference point of the prosthetic knee joint is arranged at a predetermined offset above the height of the knee gap of the intact limb.
